(19) United States
(12) Patent Application Publication
(10) Pub. No.: US 2015/0010910 A1
(43) Pub. Date: Jan. 8, 2015

(54) BIOMARKERS FOR AGE-RELATED MACULAR DEGENERATION (AMD)

(71) Applicant: UCL BUSINESS PLC, London (GB)

(72) Inventor: John Robert Watson Yates, Cambridge (GB)

(21) Appl. No.: 14/380,599

(22) PCT Filed: Feb. 21, 2013
(86) PCT No.: PCT/GB2013/050420

§ 371 Date: Aug. 22, 2014

(30) Foreign Application Priority Data
Feb. 21, 2012 (GB) 1202944.3

(51) Int. Cl. C12Q 1/68

(52) U.S. Cl. CPC C12Q 1/6883; C12Q 2600/156
USPC 435/6.11; 536/23.1

(57) ABSTRACT

A method for identifying a risk profile for age-related macular degeneration (AMD) or choroidal neovascularisation (CNV) in a subject, comprising identifying the nucleotide at one or more of the following positions: rs2071277, rs12153855 and rs9391734, or a proxy of any of those sites, in a sample obtained from the subject.

### FIELD OF THE INVENTION

The invention relates to biomarkers for identifying a predisposition of a subject to or the likelihood of a subject having age-related macular degeneration (AMD) and related conditions. The biomarkers are genetic biomarkers, specifically the presence of particular single nucleotide polymorphisms (SNPs). The biomarkers may be used in isolation or in combination with other biomarkers of AMD to establish whether a subject is likely, or unlikely, to have or develop AMD.

### BACKGROUND TO THE INVENTION

Age-related macular degeneration (AMD) is the commonest cause of blindness in Western populations {Bunce, 2010 39; Klein, 2011 40}, reducing the quality of life of tens of millions of older people worldwide. It affects the macular region of the retina which has a high density of photoreceptors for detailed central vision. Early in the disease deposits called drusen form along Bruch's membrane that separates the retinal pigment epithelium (RPE) from the underlying choroid {Jager, 2008 41}. The later stages of the disease are characterised by focal atrophy of the RPE and overlying photoreceptors (geographic atrophy, GA) and/or growth of new blood vessels from the choroid through Bruch's membrane into the RPE (choroidal neovascularisation, CNV) {Jager, 2008 41}. Both of these processes can result in loss of central vision.

Susceptibility to AMD is influenced by age, environmental and genetic factors {Jager, 2008 41}. Smoking is the most important environmental risk factor {Chakravarthy, 2010 42}. Striking progress has been made in understanding the genetics of AMD {Deangelis, 2011 43}. Common sequence variants in the complement pathway genes CFH, C2-CFB and C3 are established risk factors {Khandhadia, 2011 44} and there is another risk locus in the vicinity of CFI {Cipriani, 2011 32}. This and other evidence points to activation of the alternative complement pathway as an important component of the pathogenesis of AMD {Khandhadia, 2011 44}. Variants at the ARMS2-HTRA1 locus are strongly associated with AMD {Jakobsdottir, 2005 109; Rivera, 2005 106}, but the mechanism is uncertain {Deangelis, 2011 43}. More recently reported risk loci at LIPC {Chen, 2010 34; Neale, 2010 33; Yu, 2011 35}, CETP {Chen, 2010 34; Neale, 2010 33; Yu, 2011 35}, TIMP3-SYN3 {Chen, 2010 34; Neale, 2010 33; Yu, 2011 35} and VEGFA {Yu, 2011 35} implicate lipid metabolism, matrix homeostasis and control of angiogenesis as additional factors in the pathogenesis of AMD. The known genetic risk variants do not account for all the heritability of AMD. The inventor has therefore carried out a genome-wide association study (GWAS) in the UK population to identify additional susceptibility loci. The inventor has identified biomarkers that appear to be unrelated to known biomarkers of AMD. Surprisingly, the markers show association with both of the later forms of AMD: CNV and GA. Accordingly, the markers can be used either in isolation or in combination to diagnose a predisposition of a subject to AMD or predict the likelihood of a subject having AMD.

### SUMMARY OF THE INVENTION

The invention provides a number of SNPs that the inventor has found to be associated with having an increased risk of AMD or CNV.

There is provided a method for identifying a risk profile for AMD or CNV in a subject, comprising identifying the nucleotide at one or more of the following positions: rs2071277, rs12153855 and rs9391734, in a sample obtained from the subject.

Preferably the method includes the step of identifying the nucleotide at one or more of the following positions:

rs2071277; rs12153855 and rs9391734.

Preferably the method includes the step of identifying the nucleotide at position rs2071277; and/or the nucleotide at one of position rs12153855 and position rs9391734.

When the method is for identifying an increased risk of having or developing CNV, the method preferably includes the step of identifying the nucleotide at position rs2071277.

Preferably the method also includes the step of identifying the nucleotide at position rs541862.

The term “risk profile” preferably means the likelihood of a subject having or developing AMD or CNV. The presence of one, some or any of the following nucleotides is preferably indicative of the subject having an increased likelihood of having or developing AMD or CNV: nucleotide G or C at position rs2071277; nucleotide G or C at position rs12153855 and nucleotide A or T at position rs9391734. The presence of the nucleotide G at position rs541862 is preferably indicative of the subject having a normal or decreased risk of having or developing AMD. The terms “increased”, “normal” and “decreased” when referring to risk preferably mean that the subject has that risk level when compared with the risk associated with an individual having a different nucleotide at the position concerned. For example, a subject having nucleotide C at position rs2071277 has a higher risk of having or developing AMD than a subject having nucleotide T at that position. A subject having nucleotide C at position rs12153855 has a higher risk of having or developing AMD than a subject having nucleotide T at that position.

The method may also, or alternatively comprise identifying the nucleotide at any proxy for any of the positions mentioned.

Identification of the nucleotides at the various positions may be carried out by methods known in the art, such as those discussed in the Examples. In particular, polymerase chain reaction (PCR) based methods comprising amplifying the region of DNA comprising the polymorphism may be used. Such methods include “Amplifluor” and “TaqMan” assays, which are well known in the art.

The term AMD is well known in the art. In early stages of the disease, drusen form along Bruch's membrane which separates the retinal pigment epithelium (RPE) from the underlying choroid. The later stages of the disease include geographic atrophy (GA) and choroidal neovascularisation (CNV). Whilst the major cause of CNV is AMD, it is also seen in other conditions, such as polypoidal choroidal vasculopathy (PCV). Accordingly, the method is useful for identifying a risk profile of PCV.

Position rs2071277 is in the NOTCH4 gene, which encodes one of the four cell surface receptors of the Notch signalling pathway. Position rs12153855 is found in the TNXB gene, which encodes the extracellular matrix protein tenascin-X found in the macular Bruch's membrane/choroid complex. Its proxy, at position rs9391734, is the FKBPL gene, which encodes the FKBP-like protein belonging to the family of FK506 binding proteins. It has a role in intracellular signalling.

The subject is preferably a mammal, especially a primate, particularly a human. Alternatively, the subject may be a rodent, especially a rat or a mouse. Where the subject is not a human, the method may comprise identifying a SNP in a gene orthologous to NOTCH4, TNXB or FKBPL, especially identifying the nucleotide at a position equivalent to one or more of rs2071277, rs12153855 or rs9391734.

The sample may be any sample using which an analysis of the subject's genetic material may be made. For example, it may be a blood sample, a buccal swab or saliva sample or any other source of constitutional DNA.

The method may further comprise identifying the nucleotides at other sites known to be associated with AMD or CNV, such as sites within the C3 gene, especially at position rs2230199, and at the following genetic loci: ARMS2-HTRA1, CFH, C2-CFB, VEGFA, CCDC109B-PLA2G12A-CFI, LIPC, CETP, SYN3-TIMP3 and ND2. In particular, the method may comprise identifying the nucleotide at any of the sites given in Table 2.

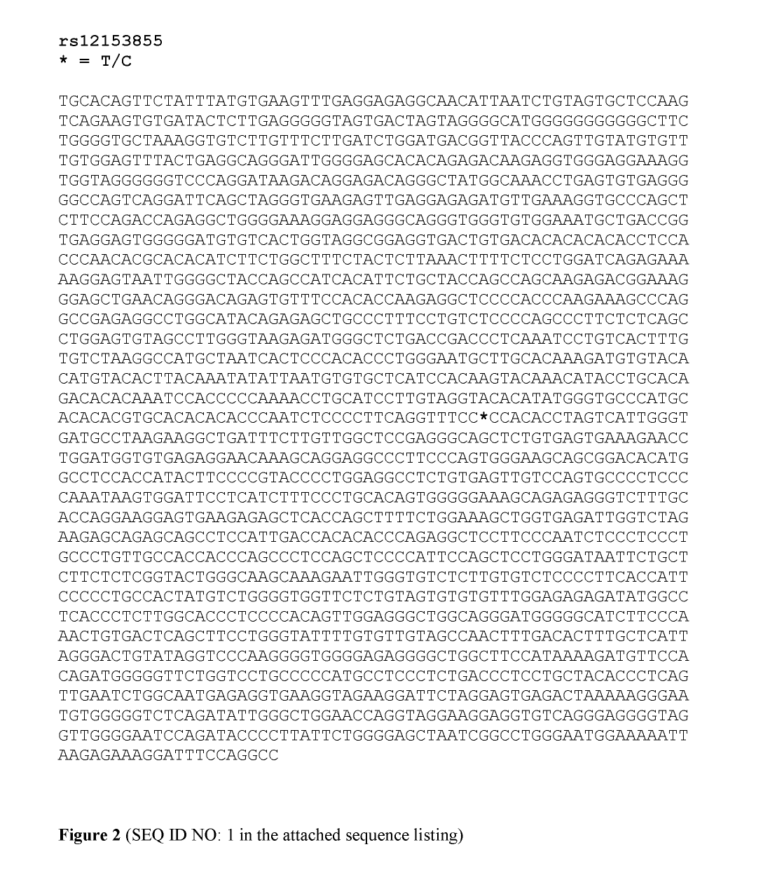

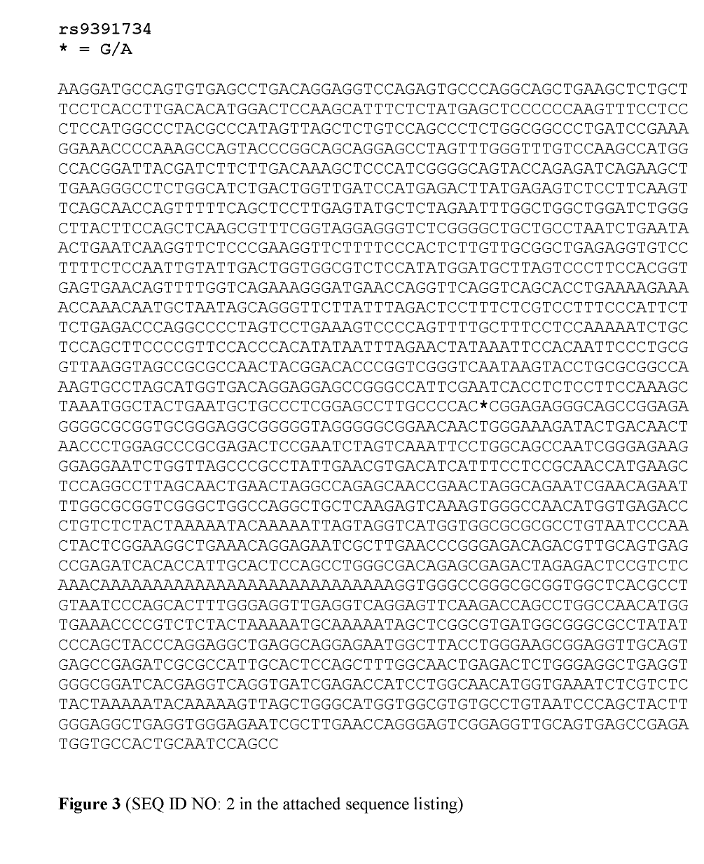

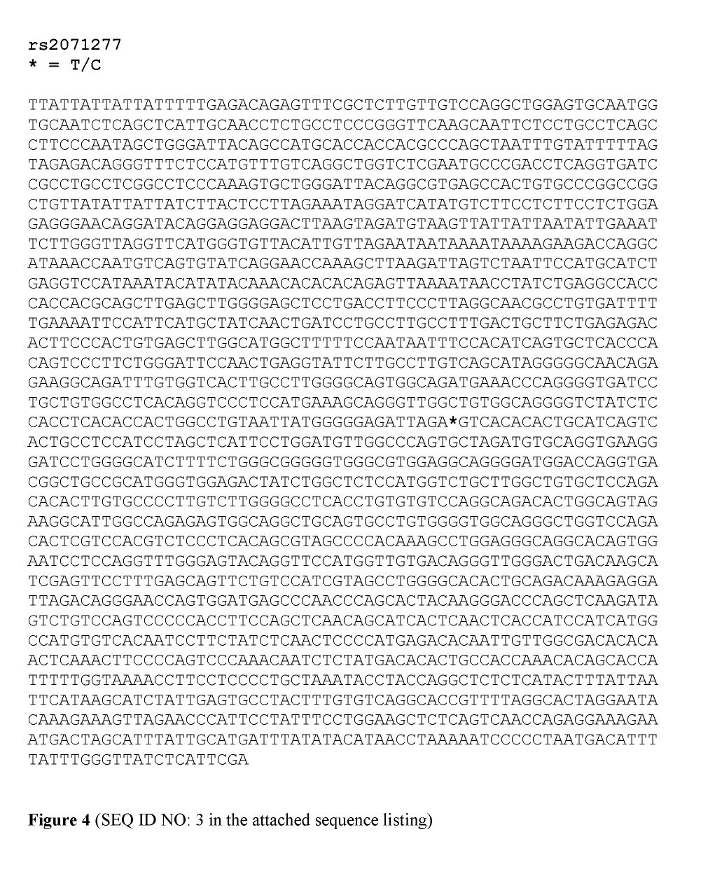

Also provided is an isolated nucleic acid comprising, preferably consisting of between about 15 and 2000 contiguous nucleotides from the sequence shown in FIG. 4 (SEQ ID NO: 3) and which includes position rs2071277. Further provided is an isolated nucleic acid comprising, preferably consisting of between about 15 and 2000 contiguous nucleotides from the sequence shown in FIG. 2 (SEQ ID NO: 1) and which includes position rs12153855. Additionally provided is an isolated nucleic acid comprising, preferably consisting of between about 15 and 2000 contiguous nucleotides from the sequence shown in FIG. 3 (SEQ ID NO: 2) and which includes position rs9391734.

Also provided is an isolated nucleic acid which hybridises to part or all of a polynucleotide containing, preferably consisting of, between about 15 and 2000 contiguous nucleotides from the sequence shown in FIG. 4 (SEQ ID NO: 3) and which includes position rs2071277. Further provided is an isolated nucleic acid which hybridises to part or all of a polynucleotide containing, preferably consisting of, between about 15 and 2000 contiguous nucleotides from the sequence shown in FIG. 2 (SEQ ID NO: 1) and which includes position rs12153855. Additionally provided is an isolated nucleic acid which hybridises to part or all of a polynucleotide containing, preferably consisting of, between about 15 and 2000 contiguous nucleotides from the sequence shown in FIG. 3 (SEQ ID NO: 2) and which includes position rs9391734.

Preferably the isolated nucleic acid is a probe or primer. Preferably the isolated nucleic acid is between 8 and 100 bases in length.

The invention also provides a kit for identifying a risk profile of AMD or CNV in a subject, the kit comprising one or more of the isolated nucleic acids of the invention. Preferably it comprises at least two of the isolated nucleic acids, especially an isolated nucleic acid which hybridises to a polynucleotide containing between about 15 and 2000 contiguous nucleotides from the sequence shown in FIG. 4 (SEQ ID NO: 3) and which includes position rs2071277 and one or both of an isolated nucleic acid which hybridises to a polynucleotide containing between about 15 and 2000 contiguous nucleotides from the sequence shown in FIG. 2 (SEQ ID NO: 1) and which includes position rs12153855 and an isolated nucleic acid which hybridises to a polynucleotide containing between about 15 and 2000 contiguous nucleotides from the sequence shown in FIG. 3 (SEQ ID NO: 2) and which includes position rs9391734.

The kit may also comprise one or more enzymes, especially a polymerase.

Also provided is the use of a NOTCH4 transgenic animal, such an animal in which the NOTCH4 gene comprises one or more mutations, as a model of AMD or CNV. The NOTCH4 transgenic animal is preferably a rodent, especially a rat or a mouse. The animal may comprise a non-functional NOTCH4 gene, a NOTCH4 gene which contains one or more mutations, or may have had the NOTCH4 gene knocked out. Such animals are known in the art.

The invention will now be described in detail, by way of example only, with reference to the drawings:

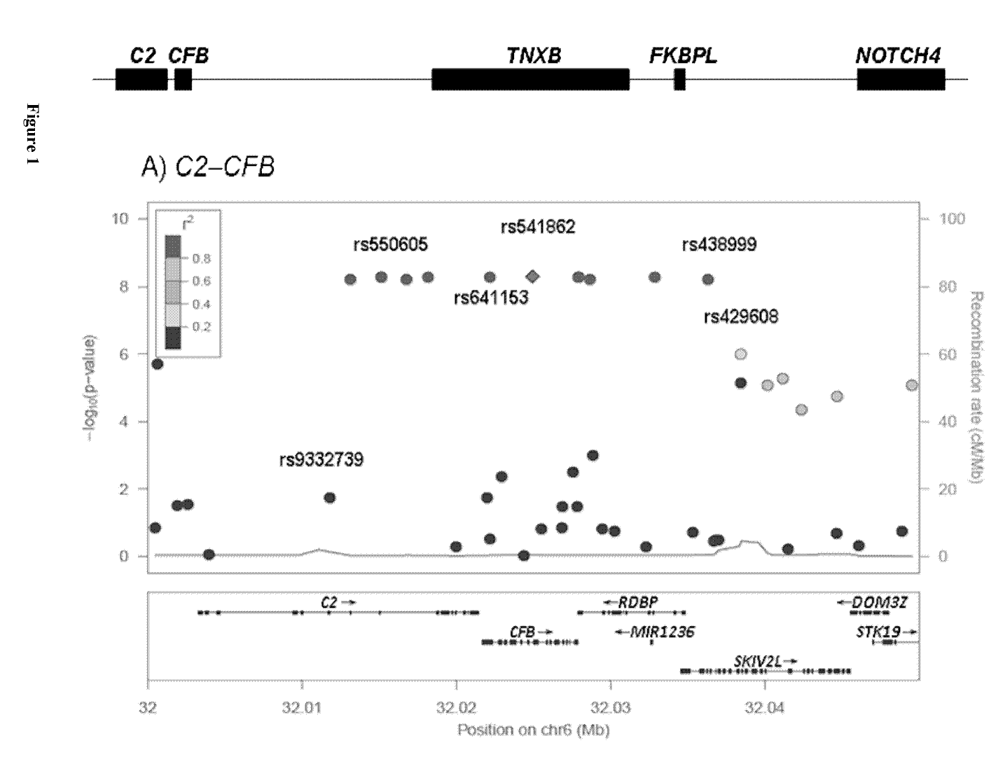

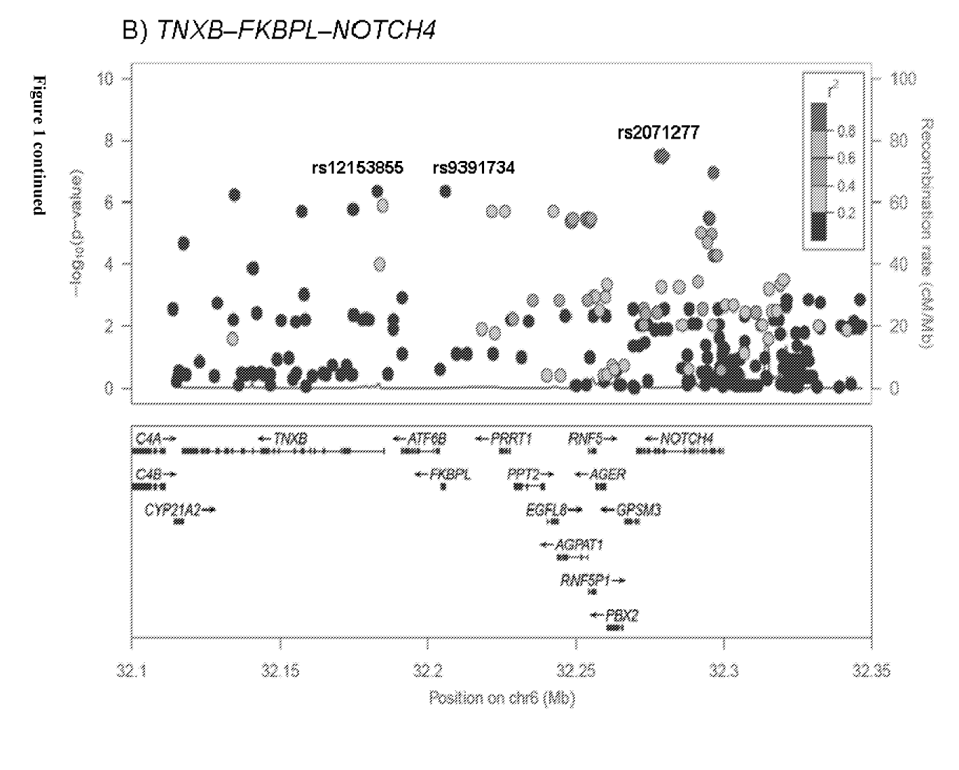

FIG. 1. Regional plots of association on chromosome 6p21.3 in the discovery samples A) for the established association signal at the C2-CFB locus B) for the newly identified association in the TNXB-FKBPL-NOTCH4 region. The most associated SNP in each region is denoted by a diamond and other SNPs by circles showing their degree of linkage disequilibrium with the most associated SNP (based on HapMap II).

FIG. 2. Portion of the sequence of TNXB gene, including the SNP identified by the inventor. SEQ ID NO: 1 in the attached sequence listing

FIG. 3. Portion of the sequence of FKBPL gene, including the SNP identified by the inventor. SEQ ID NO: 2 in the attached sequence listing

FIG. 4. Portion of the sequence of the NOTCH4 gene, including the SNP identified by the inventor. SEQ ID NO: 3 in the attached sequence listing

### EXAMPLES

### Materials and Methods

### Cases and Controls

Cases for the discovery experiment were selected from existing Cambridge and Edinburgh AMD case-control sample collections {Yates, 2007 1}. All cases selected for the GWAS had at least one eye affected by choroidal neovascularisation and/or geographic atrophy. For cases genotyped using the 300 k platform, preference was given to cases with early onset. Control genotyping data was obtained from the British 1958 Birth Cohort (58BC) {Barrett, 2009 16; Power, 2006 17}. For cases genotyped using the 300 k platform, controls matched by geographic area were selected from the 58BC subjects used in the Wellcome Trust Case Control Consortium study {Wellcome Trust Case Control Consortium, 2007 8}. For cases genotyped using the 550 k platform, controls matched by geographic area were selected from the 58BC subjects genotyped as controls for the Type 1 Diabetes Genetics Consortium (T1DGC) study {Barrett, 2009 16}. The replication studies used cases and controls from the Cambridge and Edinburgh sample collections and from case control collections from London and Southampton in the UK. All the subjects used in this research came from studies that followed the tenets of the Declaration of Helsinki and had appropriate ethical approval. All participants gave informed written consent.

Cambridge AMD Study subjects: cases and controls were recruited from ophthalmic clinics in London, the South East of England and the North West of England between 2002 and 2006 {Yates, 2007 1}. Almost all the controls were the spouses or partners of index cases and the remainder were friends of cases. All subjects described themselves as “white” rather than “other” on a recruitment questionnaire. Subjects were examined by an ophthalmologist and health, lifestyle and smoking data were collected. All subjects had colour, stereoscopic fundus photography of the macular region and the images were graded at the Reading Centre, Moorfields Eye Hospital, London using the International Classification of Age-related Maculopathy and Macular Degeneration {Bird, 1995 9}.

Edinburgh AMD Study subjects: the majority of cases and controls were recruited from ophthalmic clinics in Edinburgh, Dundee and Inverness between 2004 and 2006 {Yates, 2007 1}. Controls were predominantly recruited from cataract clinics in the same centres but some spouses were also included. Cases and controls were all examined by an ophthalmologist and visual acuities, health, lifestyle and smoking data were collected. Members of the 1921 Lothian Birth Cohort {Deary, 2004 19} were examined by an ophthalmologist and those found to have age-related macular degeneration and a similar number of unaffected controls from the Cohort were also included in the study. All cases and controls had colour, stereoscopic fundus photography of the macular region and images were graded by an ophthalmologist working on the study using the International Classification of Age-related Maculopathy and Macular Degeneration {Bird, 1995 9}; for validation 100 cases and controls were independently graded at the Moorfields Reading Centre (kappa statistic=0.84).

London AMD Study subjects: cases were recruited from Moorfields Eye Hospital, London. Controls were recruited from spouses, partners or friends of cases, or were from local residential homes for the elderly within 8 km of the hospital. All subjects were of Caucasian descent. Cases were examined by an ophthalmologist and had colour, stereoscopic fundus photography of the macular region with grading of the photographs at the Reading Centre, Moorfields Eye Hospital according to the International Classification of Age-related Maculopathy and Macular Degeneration {Bird, 1995 9}. Cases were excluded if they had retino-choroidal inflammatory disease, diabetic retinopathy, branch retinal vein or artery occlusion or any other cause of visual loss other than amblyopia. The ophthalmic examination included Snellen acuity, slit-lamp examination and biomicroscopic fundoscopy. Auto-fluorescence images were taken of the macula and fluorescein angiography was performed when choroidal neovascularisation was suspected. For patients presenting with visual dysfunction in the second eye, retrospective data was gathered from hospital records concerning previous acuities. Moreover, any color images or fluorescein images relating to previous visual loss were located from the hospital archive. All images were digitized. Controls had the same evaluation as cases and were excluded if they had drusen >63 μm or more extensive evidence of age-related macular degeneration. Each participant was interviewed specifically for the study, and a family history, smoking history and other medical history was taken.

Southampton subjects: cases were ascertained through the Southampton Eye Unit or research clinics in Guernsey, UK. Controls were spouses/partners of AMD cases, patients attending eye clinics with unrelated eye disease or their spouses/partners. All participants were white and over 55 years of age. All cases and controls were evaluated by an experienced retinal specialist and had dilated eye examination. Phenotyping was based on the Age-Related Eye Disease Study (AREDS) classification system {The Age-Related Eye Disease Study Research Group, 2001 20}, in cases taking into account the results of fundus photography and fluorescein angiography and in controls based on clinical findings alone.

### Genotyping

Genotyping of cases for the discovery experiment used genomic DNA extracted from peripheral blood leucocytes. Cases were typed either at the British Heart Foundation Glasgow Cardiovascular Research Centre using the Illumina Infinium HumanHap300 BeadChip or at the Wellcome Trust Clinical Research Facility, Edinburgh using the Illumina Infinium HumanHap550v3 BeadChip according to the manufacturer's protocols. Controls from the 1958 Birth Cohort had been previously genotyped using the Illumina Infinium HumanHap550v1 BeadChip (58BC controls) {Wellcome Trust Case Control Consortium, 2007 8} and the Illumina Infinium HumanHap550v3 BeadChip (T1DGC controls) {Barrett, 2009 16}.

The replication studies used genomic DNA extracted from peripheral blood. Genotyping was carried out by either the Medical Research Council Human Genetics Unit (MRC HGU) in Edinburgh, the Centre National de Génotypage (CNG) in France or the commercial company KBioscience. At MRC HGU and CNG genotyping was carried out using TaqMan SNP Genotyping Assays (Applied Biosystems, Foster City, USA) according to the manufacturer's protocols. KBioscience (http://www.kbioscience.co.uk) used their own proprietary KASP SNP genotyping system, a competitive allele-specific PCR incorporating a fluorescent resonance energy transfer quencher cassette (see http://www.kbioscience.co.uk/reagents/KASP.html).

### Statistical Analysis

### Genotype and Sample Quality Control

Genotypes were called using the genotyping module made available in the Illumina GenomeStudio 2009.2 software that incorporates the GenTrain 2.0 clustering algorithm. Given the different platforms used in the 300 k analysis, only the overlap set of genotyped SNPs was used in the analysis. SNPs were excluded if they presented a minor allele frequency (MAF) <0.05, or a departure from Hardy Weinberg Equilibrium (HWE) at P<10⁻⁵ in controls, or a call rate <0.975 either in cases or in controls in the 300 k and 550 k datasets separately. Genotype cluster plots were visually inspected for each putative association and results were discarded unless all cluster plots were considered clearly separated. Samples were removed if they presented inconsistencies between reported gender and gender determined by heterozygosity on the X chromosome or showed inter-individual genetic relatedness. To control for possible population stratification, cases were stratified according to their reported geographic origin in four strata (London; North Midlands, Eastern, Southeastern and Southern England; Northwestern England; and Scotland) and only controls from matching geographic regions were used.

### Imputation

Autosomal imputation was performed using the R package snpStats from the BioConductor Project (http://www.bioconductor.org) and CEU HapMap II samples as reference dataset. The imputation method has been described previously {Wallace, 2010 73}. Briefly, for the 300 k and the 550 k array separately, HapMap SNPs were divided into those that passed quality control steps in the study (X) and those that were not typed or failed quality control (Y). Linear regression models were used to predict each SNP in Y from nearby SNPs in X. R² was used as a measure of accuracy of the imputation.

### Discovery and Replication Association Analyses

Single SNP association analyses were carried out using the R package snpStats (http://www.bioconductor.org). For both discovery and replication analyses, single SNP association was evaluated using the Mantel extension of the 1-degree-of-freedom (df) trend test {Mantel, 1963 18}, that is a stratified version of the Cochrane-Armitage test, to take into account the geographic stratification of the samples. Results from the 300 k and 550 k array were combined by pooling the respective single SNP 1-df score statistics and corresponding combined P-values were obtained from a 1-df chi-square distribution. Presence of heterogeneity between the 300 k and 550 k association results was evaluated through a 1-df chi- square test. Presence of residual population structure was assessed by evaluating the genomic inflation factor (λ) {Devlin, 1999 30} and examining the quantile-quantile plots of the genome-wide P-values before and after exclusion of the established AMD susceptibility loci. SNPs showing association at P<10⁻⁶ in regions not previously reported were selected for follow-up in the replication samples.

### Conditional and Haplotype-Based Association Analyses

To detect secondary independent signals within associated regions, the inventor performed stepwise logistic regression models stratified by array and geographic origin after controlling for the strongest associated genotyped variants at each region added to the models in a forward fashion (R package snpStats, http://www.bioconductor.org).

Haplotype-based analysis was carried out in PLINK v1.07 {Purcell, 2007 4}. Array information and geographic origin were added as covariates in the analysis. Only haplotypes with overall frequency >1% were considered. The ORs and the corresponding CIs were estimated using the commonest haplotype as reference.

### Results

The inventor conducted a genome wide association study in the UK population obtaining genotypes for 893 cases and 2199 controls that passed quality control metrics (Table 1). Subjects were typed with either the Illumina 300 k array (150 cases and 601 controls) or the Illumina 550 k array (743 cases and 1598 controls). Quality control criteria were not met for 24125 SNPs in the 300 k array and 68531 in the 550 k array. A small number of additional SNPs were excluded after visual inspection of their cluster plots so that subsequent analyses were based on 286135 and 488867 genotyped SNPs in the 300 k and 550 k arrays respectively. The inventor imputed variants using the CEU HapMap II reference panel and combined results from the two arrays following imputation for a total of 2273677 SNPs. Unless otherwise specified, the results discussed here did not show evidence of heterogeneity between the two platforms. The genomic inflation factor (λ) was 1.03 and the quantile-quantile plots showed no widespread departure from the expected distribution of P-values. Therefore, no further correction for inflation was applied to the test statistics. Replication studies were conducted on a sample of up to 1411 advanced AMD cases and 1431 controls.

### Previously Reported Definite and Probable AMD Associated Loci

Association at genome-wide significance level (i.e. P<5×10⁻⁸) was observed at the well established AMD susceptibility loci ARMS2-HTRA1 (rs10490924/rs2284665, P=2.7×10⁻⁷²), CFH (rs10801555, an almost perfect proxy for rs1061170, P=2.3×10⁻⁴⁷) and C2-CFB (rs541862, a perfect proxy for rs641153, P=5.2×10⁻⁹). At the CFH locus, an analysis conditional on rs10801555 confirmed a second independent association with rs1329428 (P=1.1×10⁻¹⁰). For the reported independent signal at SNP rs9332739 in C2 {Maller, 2006 31}, the inventor observed weak statistical support with P=0.02 and no association when conditioning on rs541862 (P=0.78). These results are presented in Table 2 together with association signals at other previously reported definite or probable AMD risk loci.

At the LIPC locus, the reported SNP rs493258 {Chen, 2010 34; Neale, 2010 33} showed weak evidence of association (P=0.04) but in the same direction as previously reported (OR=0.89 and 95% CI=0.79-0.99 for allele T). The association with the reported functional variant rs10468017 in this gene {Yu, 2011 35} (R²=0.42) was unconvincing (P=0.11, OR=0.91 and 95% CI=0.80-1.03 for allele T). The inventor also found an association signal for the SNP rs943080 at the VEGFA locus {Yu, 2011 35} (P=1.6×10⁻³, a perfect proxy for the reported SNP rs4711751, OR=1.20 and 95% CI=1.07-1.35 for allele T), but no association with the other reported SNP rs833069 {Galan, 2010 10} (P=0.18, OR=0.92 and 95% CI=0.82-1.04). At the CFI locus, evidence of association was found with the previously reported SNP rs7690921 in CCDC109B {Neale, 2010 33} (P=3.6×10⁻³, OR=1.19 and 95% CI=1.06-1.34 for allele T), but not for rs10033900 {Fagerness, 2009 38} (P=0.22) and rs2285714 {Chen, 2010 34} (P=0.92). The inventor did not find support for the previously reported association with variants at CETP (rs3764261, P=0.26) {Yu, 2011 35} or SYN3-TIMP3 (rs9621532, P=0.48) {Yu, 2011 35}.

At the APOE locus, the two SNPs that determine the E2/E3/E4 APOE alleles (i.e. rs429358 and rs7412) could not be imputed. However, there was evidence of association with rs2075650 (P=3.1×10⁻⁶) in the same region, albeit with some degree of heterogeneity between the 300 k and 550 k arrays (P=0.06). SNP rs2075650 lies within TOMM40 and is in modest linkage disequilibrium (LD) with rs429358 (R²=0.2). The inventor genotyped rs2075650 and APOE SNP rs429358 in the replication samples) and confirmed the known protective effect of the C allele of rs429358 (P=2.8×10⁻⁶; heterogeneity P=0.04; OR=0.68; 95% CI=0.58-0.80) and the association with rs2075650 (P=0.0038; OR=0.79 and 95% CI=0.67-0.93 for allele G), although some heterogeneity across the follow-up samples was observed (P=0.02) (Table 3). However, the association with rs2075650 became non-significant after conditioning on rs429358 (P=0.64).

### Novel Loci

Using a threshold of P<10⁻⁶ and excluding previously known associations, the inventor identified four novel associated SNPs. For one of these, rs12231166 (P=6.2×10⁻⁷) at 12q23.1, the inventor genotyped the proxy rs476497 (R²=0.92) in the replication samples, but found no evidence of association (replication P=0.87, combined P=) (Table 3). The other novel SNPs were rs2071277 in NOTCH4 (P=3.2×10⁻⁸), rs12153855 in TNXB (P=4.3×10⁻⁷) and the imputed SNP rs9391734 (a perfect proxy for rs12153855) in FKBPL (P=4.3×10⁻⁷), all located at 6p21.3 and about 250 kb from C2-CFB (FIG. 1). These SNPs are in low LD with rs541862 (R²=0.02 and R²=0.01 for rs2071277 and rs12153855/rs9391734 respectively), the SNP in CFB which has a well established association with AMD.

Conditioning for the effect of rs541862 (P=5.2×10⁻⁹) could not eliminate evidence of association for several SNPs spanning the TNXB-NOTCH4 locus (FIG. 1-B). Together with rs541862 in CFB, the inventor selected for follow-up rs12153855 in TNXB which showed a conditioned P=9.0×10⁻⁶ and rs2071277 and rs3132946 in NOTCH4 for which the inventor observed some residual evidence of association after controlling for both rs541862 and rs12153855 (P=3.8×10⁻³ and P=3.5×10⁻³ respectively). Corresponding replication results are summarised in Table 3. As expected, the known protective association at the G allele of rs541862 was confirmed (P=8.8×10⁻¹⁷; OR=0.53; 95% CI=0.46-0.62 when combining all samples). Association at both rs2071277 and rs12153855 was consistently corroborated across the two replication samples (P=3.8×10⁻⁵ and 3.0×10⁻⁴ respectively) and reached genome-wide significance when combining all samples (P=2.0×10⁻¹¹; OR=1.30; 95% CI=1.20-1.41 for allele G and P=1.3×10⁻⁹; OR=1.44; 95% CI=1.28-1.63 for allele C respectively). Repeating the conditional analysis the inventor found that both rs12153855 in TNXB and rs2071277 in NOTCH4 conferred independent risk beyond the known signal at the C2-CFB locus as shown in Table 4. Either conditioning on rs541862 and rs12153855 or on rs541862 and rs2071277 did not eliminate the effect at the third SNP with a conditioned P-value of 9.0×10⁻⁴ for rs2071277 and 2.5×10⁻⁴ for rs12153855.

To further characterize the association in this region, the inventor carried out a haplotype analysis of rs541862, rs12153855 and rs2071277. Four haplotypes at frequency >1% accounted for 98.9% of the total haplotypes observed (Table 5). The most frequent haplotype TTT was used as reference to calculate ORs. Distinct levels of AMD risk with non-overlapping confidence intervals were associated with these four haplotypes (P=9.1×10⁻²³). The known protective haplotype (CTT) tagged by the C allele of rs541862 was present in 9.4% of controls and 5.1% of cases after combining all samples. The other two haplotypes TTC and TCC that do not harbour the protective C allele of rs541862 significantly differed from the baseline haplotype TTT with OR=1.14 (95% CI=1.05-1.25) and OR=1.47 (95% CI=1.29-1.67) respectively.

### Secondary Analyses

Genome-wide association analyses were repeated on the sub-samples of subjects affected by CNV only and GA only. Only SNPs at the CFH and ARMS2-HTRA1 loci showed association at a genome-wide level of significance in both subgroup analyses. Evidence of association was observed at the newly identified SNPs rs12153855/rs9391734 in TNXB-FKBPL (P=5.0×10⁻⁴, OR=0.70, 95% CI=0.58-0.86 and P=5.0×10⁻⁶, OR=0.51, 95% CI=0.38-0.68 in the CNV only and GA only subgroup analysis respectively). The evidence of association at SNP rs2071277 in NOTCH4 was stronger in the CNV only subsample than in the GA only subsample (P=3.5×10⁻⁶, OR=0.73, 95% CI=0.64-0.84 and P=0.07, OR=0.82, 95% CI=0.66-1.01 respectively), but with no statistically significant difference between the two subgroup analyses (P=0.81).

### Discussion

The inventor has carried out the first genome-wide association study of AMD in the UK population. As expected the inventor found strong association with the well established susceptibility loci AMRS2-HTRA1, CFH, CFB-C2, C3 and CFI {Deangelis, 2011 43; Khandhadia, 2011 44}. There was also evidence of association for more recently reported risk loci at VEGFA {Yu, 2011 35} and LIPC {Chen, 2010 34; Neale, 2010 33; Yu, 2011 35} but not CETP or TIMP3-SYN3 {Chen, 2010 34; Neale, 2010 33; Yu, 2011 35}.

At the CFI locus the strongest association was with a SNP in the neighbouring CCDC109B gene as reported by others {Neale, 2010 33} rather than with the originally reported SNP rs10033900 closer to CFI {Fagerness, 2009 38}. This is unsurprising as the inventor has previously reported lack of association with rs10033900 in two independent UK case-control samples {Cipriani, 2011 32} and it was a subset of cases from these studies that was used in their GWAS. The other reported association at this locus with rs2285714 in PLA2G12A {Chen, 2010 34} showed no evidence of association in their discovery analysis. Further studies are needed to clarify the origin of the association signal in this region.

Previous studies have shown an association between AMD and the APOE E2/E4 protein polymorphism {McKay, 2011 36}. The arrays used in their GWAS did not include the two SNPs that determine these variants and they could not be imputed. However, the inventor did observe a strong association with rs2075650 in the neighbouring TOMM40 gene which was confirmed in the replication studies. Conditional analysis showed that this signal was secondary to the association with E4, providing further support for the association of APOE with AMD {McKay, 2011 36}.

The association at the CFH locus on chromosome 1q32 was extensive, as found in other GWAS surveys {Chen, 2010 34; Neale, 2010 33; Yu, 2011 35}. It spanned a megabase of DNA encompassing many genes. Conditional analysis of the region identified two independent association signals, one characterised by the SNP rs10801555 (an almost perfect proxy for rs10611760, Y402H) and the other by rs1329428 (a proxy for rs1410996). These two independent association signals have been reported previously {Mailer, 2006 31}.

On chromosome 10q26 there was strong association spanning the AMRS2 and HTRA1 genes as found by others {Chen, 2010 34; Neale, 2010 33; Yu, 2011 35}, but high LD prevented further localisation of the association signal.

On chromosome 6p21.3 there was strong association with rs541862, a perfect proxy for a nonsynonymous change R32Q (rs641153) in CFB which has a well established association with AMD, the minor allele being protective {Mailer, 2006 31}. This was one of several strongly associated SNPs spanning C2, CFB, RDBP and SKIV2L in a region of high LD (FIG. 1-A) which makes the dissection of the statistical association difficult. Support for rs641153 as the causal variant comes from functional studies showing that the 32Q variant of CFB has reduced activity compared to 32R because of weaker binding to C3b {Montes, 2009 72}. Another associated SNP from this region, rs438999 (R151Q) in the SKIV2L has also been suggested as a causal variant {McKay, 2009 50} and a protective effect of rs429608 in the same gene has been reported {Kopplin, 2010 3}. The data showed evidence of association with both rs438999 and rs429608, but conditional analysis did not provide support for an association at SKIV2L independent of the signal from rs541862 in CFB.

On chromosome 6p21.3 the inventor also found strong association reaching genome wide significance for the SNP rs2071277 in the NOTCH4 gene some 250 kb from CFB. Conditional analysis confirmed this was independent of the C2-CFB signal represented by rs541862 and also provided support for a second independent association with rs12153855 in the intervening TNXB gene. Only four haplotypes defined by these three SNPs were observed (Table 5), including the protective haplotype CTT characterised by a C allele at rs541862 which is the well established protective association reported for C2-CFB. The other three haplotypes had an T allele at rs541862. The commonest of these was TTT which the inventor designated as the reference haplotype. The next most common haplotype TTC was associated with increased risk for AMD giving an odds ratio of 1.14 (95% CI 1.05-1.25); the fourth haplotype TCC was associated with higher risk, the odds ratio being 1.47 (95% CI 1.29-1.67).

Both rs12153855 in TNXB and rs2071277 in NOTCH4 are intronic and unlikely to be causal variants. The SNP rs12153855 has a perfect proxy rs9391734 in the 5′ untranslated region of the neighbouring gene FKBPL which might have functional significance. So the inventor has identified novel associated variants but do not have evidence to pinpoint the causal variants responsible for these associations, as is the case with other susceptibility loci for AMD and many other complex diseases. All three genes could play a role in the pathogenesis of AMD as reviewed below. However, given the extensive LD across this region and their incomplete SNP coverage, the inventor cannot completely exclude the possibility that the causal variants are in other genes. Other disease associations have been reported for this region of chromosome 6 and notably an association with dementia and variants near NOTCH4 and AGER {Bennet, 2011 57}.

TNXB encodes the extracellular matrix protein tenascin-X which is present in the macular Bruch's membrane/choroid complex {Yuan, 2010 58}. Various functions have been attributed to tenascin-X which could have relevance to AMD. It has been shown to modulate collagen deposition {Mao, 2002 59}. There is also evidence that it modulates the mechanical properties of collagen networks {Margaron, 2010 60} and is involved in the maturation and/or maintenance of collagen and elastin networks {Egging, 2006 61}; both collagen and elastin are present in the Bruch's membrane. In addition, forms of tenascin-X have been shown to bind to VEGFA and VEGFB and enhance their pro-angiogenic activity {Ikuta, 2001 62}.

FKBPL encodes the FKBP-like protein which belongs to the family of FK506 binding proteins and has a role in intracellular signalling {McKeen, 2011 63}. Of particular interest in the context of AMD, FKBPL also appears to have a separate role in controlling angiogenesis as a secreted protein that inhibits endothelial cell migration, tubule formation and angiogenesis through a CD44-mediated pathway {Valentine, 2011 64}.

NOTCH4 encodes one of the four cell surface receptors of the Notch signalling pathway which has a key role in cell differentiation decisions across many cell types and at different stages of cell lineage progression {Andersson, 2011 65}. Notch signalling is important in embryonic development, the regulation of tissue homeostasis and the maintenance of stem cells. Notch signalling is particularly important in vascular development, not only in embryogenesis but also in pathological angiogenesis, prompting research on Notch pathway inhibitors as potential agents to disrupt tumour related angiogenesis for cancer therapy {Kerbel, 2008 66}.

Notch signalling plays an important role in the developing retinal vasculature by regulating the specification of endothelial cells into stalk and tip cells {Hellstrom, 2007 67}. In studies of mice, NOTCH4 is strongly expressed in retinal arterial endothelial cells {Claxton, 2004 68}. Moreover, Notch signalling has been shown to influence pathological angiogenesis in a study of laser-induced CNV in rats {Ahmad, 2011 69}. Following laser photocoagulation, inhibition of Notch signalling by intravitreal or subcutaneous administration of the gamma secretase inhibitor DAPT resulted in a substantial increase in CNV volume compared to controls; conversely, stimulation of Notch signalling by intravitreal administration of Jagged1 peptide, a Notch ligand, substantially reduced CNV volume {Ahmad, 2011 69}. In the same report evidence was presented that Notch signalling contributes to angiogenic homeostasis by providing a counterbalance to proangiogenic pathways such as that mediated by VEGF {Ahmad, 2011 69}. This led the authors to propose the Notch pathway as a potential therapeutic target for the treatment of CNV which is the severest form of AMD {Ahmad, 2011 69}. This might complement the use of anti-VEGF antibodies which is the mainstay of current treatment. The finding that SNPs at the NOTCH4 locus are associated with AMD provides support for the involvement of Notch signalling in AMD and points specifically to a role for the NOTCH4 receptor. However, if this were mediated through an alteration in angiogenic homeostasis the inventor might expect the association to be specifically with CNV. Most of the association signal does indeed come from CNV because this is the condition affecting most of the cases in the study, but the subgroup analysis suggests that there is also an association in the minority of cases with GA. This raises the possibility that other functions of the Notch pathway may be more important, such as its role in the differentiation of immune cells, including macrophages which recent evidence suggests may play a key part in the pathogenesis of AMD {Cherepanoff, 2010 70}.

In summary, this UK genome-wide association study has shown the expected association between AMD and known risk loci at ARMS2-HTRA1, CFH, C2-CFB, C3 and CFI-CCDC109B and with more recently reported risk loci at LIPC and near VEGFA. In addition the inventor has found novel associations with variants in TNXB-FKBPL and the neighbouring gene NOTCH4 on chromosome 6p21.3 which are independent of the associated SNPs at the nearby C2-CFB locus. TNXB, FKBPL and NOTCH4 are all plausible AMD susceptibility genes and the Notch signalling pathway has already been suggested as a potential therapeutic target for AMD {Ahmad, 2011 69}. Nevertheless, it is surprising that the SNP identified in the NOTCH4 gene is a marker for both GA and CNV, Notch being associated with inappropriate angiogenesis and therefore unlikely to be expected to be associated with GA.

### REFERENCES

1. Bunce, C., Xing, W., Wormald, R. (2010) Causes of blind and partial sight certifications in England and Wales: April 2007-March 2008. Eye (Lond), 24, 1692-1699.
2. Klein, R., Chou, C. F., Klein, B. E. K., Zhang, X., Meuer, S. M., Saaddine, J. B. (2011) Prevalence of Age-Related Macular Degeneration in the US Population. Arch. Ophthalm., 129, 75-80.
3. Jager, R. D., Mieler, W. F., Miller, J. W. (2008) Age-related macular degeneration. N. Engl. J. Med., 358, 2606-2617.
4. Chakravarthy, U., Wong, T. Y., Fletcher, A., Piault, E., Evans, C., Zlateva, G., Buggage, R., Pleil, A., Mitchell, P. (2010) Clinical risk factors for age-related macular degeneration: a systematic review and meta-analysis. BMC Ophthalmol., 10, 31.
5. Deangelis, M. M., Silveira, A. C., Carr, E. A., Kim, I. K. (2011) Genetics of age-related macular degeneration: current concepts, future directions. Semin. Ophthalmol., 26, 77-93.
6. Khandhadia, S., Cipriani, V., Yates, J. R., Lotery, A. J. (2011) Age-related macular degeneration and the complement system. Immunobiology, 217, 127-146.
7. Cipriani, V., Matharu, B. K., Khan, J. C., Shahid, H., Hayward, C., Wright, A. F., Armbrecht, A. M., Dhillon, B., Harding, S. P., Bishop, P. N., et al. (2012) No evidence of association between complement factor I genetic variant rs10033900 and age-related macular degeneration. Eur. J. Hum. Genet., 20, 1-2.
8. Jakobsdottir, J., Conley, Y. P., Weeks, D. E., Mah, T. S., Ferrell, R. E., Gorin, M. B. (2005) Susceptibility genes for age-related maculopathy on chromosome 10q26. Am. J. Hum. Genet., 77, 389-407.
9. Rivera, A., Fisher, S. A., Fritsche, L. G., Keilhauer, C. N., Lichtner, P., Meitinger, T., Weber, B. H. (2005) Hypothetical LOC387715 is a second major susceptibility gene for age-related macular degeneration, contributing independently of complement factor H to disease risk. Hum. Mol. Genet., 14, 3227-3236.
10. Chen, W., Stambolian, D., Edwards, A. O., Branham, K. E., Othman, M., Jakobsdottir, J., Tosakulwong, N., Pericak-Vance, M. A., Campochiaro, P. A., Klein, M. L., et al. (2010) Genetic variants near TIMP3 and high-density lipoprotein-associated loci influence susceptibility to age-related macular degeneration. Proc. Natl. Acad. Sci. U.S.A., 107, 7401-7406.
11. Neale, B. M., Fagerness, J., Reynolds, R., Sobrin, L., Parker, M., Raychaudhuri, S., Tan, P. L., Oh, E. C., Merriam, J. E., Souied, E., et al. (2010) Genome-wide association study of advanced age-related macular degeneration identifies a role of the hepatic lipase gene (LIPC). Proc. Natl. Acad. Sci. U.S.A., 107, 7395-7400.
12. Yu, Y., Bhangale, T. R., Fagerness, J., Ripke, S., Thorleifsson, G., Tan, P. L., Souied, E. H., Richardson, A. J., Merriam, J. E., Buitendijk, G. H., et al. (2011) Common variants near FRK/COL10A1 and VEGFA are associated with advanced age-related macular degeneration. Hum. Mol. Genet., 20, 3699-3709.
13. Maller, J., George, S., Purcell, S., Fagerness, J., Altshuler, D., Daly, M. J., Seddon, J. M. (2006) Common variation in three genes, including a noncoding variant in CFH, strongly influences risk of age-related macular degeneration. Nat. Genet., 38, 1055-1059.
14. Galan, A., Ferlin, A., Caretti, L., Buson, G., Sato, G., Frigo, A. C., Foresta, C. (2010) Association of age-related macular degeneration with polymorphisms in vascular endothelial growth factor and its receptor. Ophthalmology, 117, 1769-1774.
15. Fagerness, J. A., Maller, J. B., Neale, B. M., Reynolds, R. C., Daly, M. J., Seddon, J. M. (2009) Variation near complement factor I is associated with risk of advanced AMD. Eur. J. Hum. Genet., 17, 100-104.
16. Kopplin, L. J., Igo, R. P., Jr., Wang, Y., Sivakumaran, T. A., Hagstrom, S. A., Peachey, N. S., Francis, P. J., Klein, M. L., SanGiovanni, J. P., Chew, E. Y., et al. (2010) Genome-wide association identifies SKIV2L and MYRIP as protective factors for age-related macular degeneration. Genes Immun., 11, 609-621.
17. McKay, G. J., Patterson, C. C., Chakravarthy, U., Dasari, S., Klaver, C. C., Vingerling, J. R., Ho, L., de Jong, P. T., Fletcher, A. E., Young, I. S., et al. (2011) Evidence of association of APOE with age-related macular degeneration—a pooled analysis of 15 studies. Hum. Mutat., 32, 1407-1416.
18. Montes, T., Tortajada, A., Morgan, B. P., Rodriguez de, C. S., Harris, C. L. (2009) Functional basis of protection against age-related macular degeneration conferred by a common polymorphism in complement factor B. Proc. Natl. Acad. Sci. U.S.A, 106, 4366-4371.
19. McKay, G. J., Silvestri, G., Patterson, C. C., Hogg, R. E., Chakravarthy, U., Hughes, A. E. (2009) Further assessment of the complement component 2 and factor B region associated with age-related macular degeneration. Invest. Ophthalmol. Vis. Sci., 50, 533-539.
20. Bennet, A. M., Reynolds, C. A., Eriksson, U. K., Hong, M. G., Blennow, K., Gatz, M., Alexeyenko, A., Pedersen, N. L., Prince, J. A. (2011) Genetic association of sequence variants near AGER/NOTCH4 and dementia. J. Alzheimers. Dis., 24, 475-484.
21. Yuan, X., Gu, X., Crabb, J. S., Yue, X., Shadrach, K., Hollyfield, J. G., Crabb, J. W. (2010) Quantitative proteomics: comparison of the macular Bruch membrane/choroid complex from age-related macular degeneration and normal eyes. Mol. Cell. Proteomics, 9, 1031-1046.
22. Mao, J. R., Taylor, G., Dean, W. B., Wagner, D. R., Afzal, V., Lotz, J. C., Rubin, E. M., Bristow, J. (2002) Tenascin-X deficiency mimics Ehlers-Danlos syndrome in mice through alteration of collagen deposition. Nat. Genet., 30, 421-425.
23. Margaron, Y., Bostan, L., Exposito, J. Y., Malbouyres, M., Trunfio-Sfarghiu, A. M., Berthier, Y., Lethias, C. (2010) Tenascin-X increases the stiffness of collagen gels without affecting fibrillogenesis. Biophys. Chem., 147, 87-91.
24. Egging, D. F., van, V., I, Starcher, B., Gijsen, Y., Zweers, M. C., Blankevoort, L., Bristow, J., Schalkwijk, J. (2006) Dermal connective tissue development in mice: an essential role for tenascin-X. Cell Tissue Res., 323, 465-474.
25. Ikuta, T., Ariga, H., Matsumoto, K. I. (2001) Effect of tenascin-X together with vascular endothelial growth factor A on cell proliferation in cultured embryonic hearts. Biol. Pharm. Bull., 24, 1320-1323.
26. McKeen, H. D., Brennan, D. J., Hegarty, S., Lanigan, F., Jirstrom, K., Byrne, C., Yakkundi, A., McCarthy, H. O., Gallagher, W. M., Robson, T. (2011) The emerging role of FK506-binding proteins as cancer biomarkers: a focus on FKBPL. Biochem. Soc. Trans., 39, 663-668.
27. Valentine, A., O'Rourke, M., Yakkundi, A., Worthington, J., Hookham, M., Bicknell, R., McCarthy, H. O., McClelland, K., McCallum, L., Dyer, H., et al. (2011) FKBPL and peptide derivatives: novel biological agents that inhibit angiogenesis by a CD44-dependent mechanism. Clin. Cancer. Res., 17, 1044-1056.
28. Andersson, E. R., Sandberg, R., Lendahl, U. (2011) Notch signaling: simplicity in design, versatility in function. Development, 138, 3593-3612.
29. Kerbel, R. S. (2008) Tumor angiogenesis. N. Engl. J. Med., 358, 2039-2049.
30. Hellstrom, M., Phng, L. K., Hofmann, J. J., Wallgard, E., Coultas, L., Lindblom, P., Alva, J., Nilsson, A. K., Karlsson, L., Gaiano, N., et al. (2007) D114 signalling through Notch1 regulates formation of tip cells during angiogenesis. Nature, 445, 776-780.
31. Claxton, S., Fruttiger, M. (2004) Periodic Delta-like 4 expression in developing retinal arteries. Gene Expr. Patterns, 5, 123-127.
32. Ahmad, I., Balasubramanian, S., Del Debbio, C. B., Parameswaran, S., Katz, A. R., Toris, C., Fariss, R. N. (2011) Regulation of ocular angiogenesis by Notch signaling: implications in neovascular age-related macular degeneration. Invest. Ophthalmol. Vis. Sci., 52, 2868-2878.
33. Cherepanoff, S., McMenamin, P., Gillies, M. C., Kettle, E., Sarks, S. H. (2010) Bruch's membrane and choroidal macrophages in early and advanced age-related macular degeneration. Br. J. Ophthalmol., 94, 918-925.
34. Yates, J. R., Sepp, T., Matharu, B. K., Khan, J. C., Thurlby, D. A. , Shahid, H., Clayton, D. G., Hayward, C., Morgan, J., Wright, A. F., et al. (2007) Complement C3 variant and the risk of age-related macular degeneration. N. Engl. J. Med., 357, 553-561.
35. Barrett, J. C., Clayton, D. G., Concannon, P., Akolkar, B., Cooper, J. D., Erlich, H. A., Julier, C., Morahan, G., Nerup, J., Nierras, C., et al. (2009) Genome-wide association study and meta-analysis find that over 40 loci affect risk of type 1 diabetes. Nat. Genet., 41, 703-707.
36. Power, C., Elliott, J. (2006) Cohort profile: 1958 British birth cohort (National Child Development Study). Int. J. Epidemiol., 35, 34-41.
37. Wellcome Trust Case Control Consortium (2007) Genome-wide association study of 14,000 cases of seven common diseases and 3,000 shared controls. Nature, 447, 661-678.
38. Bird, A. C., Bressler, N. M., Bressler, S. B., Chisholm, I. H., Coscas, G., Davis, M. D., de Jong, P. T., Klaver, C. C. W., Klein, B. E. K., Klein, R., et al. (1995) An international classification and grading system for age-related maculopathy and age-related macular degeneration. The International ARM Epidemiological Study Group. Surv. Ophthalmol., 39, 367-374.
39. Deary, I. J., Whiteman, M. C., Starr, J. M., Whalley, L. J., Fox, H. C. (2004) The impact of childhood intelligence on later life: following up the Scottish mental surveys of 1932 and 1947. J. Pers. Soc. Psychol., 86, 130-147.
40. The Age-Related Eye Disease Study Research Group (2001) The age-related eye disease study system for classifying age-related macular degeneration from stereoscopic color fundus photographs: the age-related eye disease study report number 6. Am. J. Ophthalmol., 132, 668-681.
41. Wallace, C., Smyth, D. J., Maisuria-Armer, M., Walker, N. M., Todd, J. A., Clayton, D. G. (2010) The imprinted DLK1-MEG3 gene region on chromosome 14q32.2 alters susceptibility to type 1 diabetes. Nat. Genet., 42, 68-71.
42. Mantel, N. (1963) Chi-Square Tests with One Degree of Freedom; Extensions of the Mantel-Haenszel Procedure. J. Am. Stat. Assoc., 58, 690-700.
43. Devlin, B., Roeder, K. (1999) Genomic control for association studies. Biometrics, 55, 997-1004.
44. Purcell, S., Neale, B., Todd-Brown, K., Thomas, L., Ferreira, M. A., Bender, D., Maller, J., Sklar, P., de Bakker, P. I., Daly, M. J., Sham, P. C. (2007) PLINK: a tool set for whole-genome association and population-based linkage analyses. Am. J. Hum. Genet., 81, 559-575.
45. Klein, R. J., Zeiss, C., Chew, E. Y., Tsai, J. Y., Sackler, R. S., Haynes, C., Henning, A. K., SanGiovanni, J. P., Mane, S. M., Mayne, S. T., et al. (2005) Complement factor H polymorphism in age-related macular degeneration. Science, 308, 385-389.
46. Gold, B., Merriam, J. E., Zernant, J., Hancox, L. S., Taiber, A. J., Gehrs, K., Cramer, K., Neel, J., Bergeron, J., Barile, G. R., et al. (2006) Variation in factor B (BF) and complement component 2 (C2) genes is associated with age-related macular degeneration. Nat. Genet., 38, 458-462.

### TABLES

TABLE 1
Gender, age, phenotype and geographic origin of subjects in the
discovery samples.

| | Cases | Controls |
|---|---|---|
| | (N = 893) | (N = 2199) |
| Female, N (%) | 494 (55.3) | 1117 (50.8) |
| Mean age ± SD, yr | 78.6 ± 7.5 | 44-45^{a} |
| Phenotype | | |
| Normal | | 2199 |
| CNV | 593 | |
| GA | 182 | |
| CNV and GA | 118 | |
| Geographic origin | | |
| L | 80 | 156 |
| NMESES | 548 | 1373 |
| NW | 163 | 272 |
| S | 102 | 398 |

CNV: choroidal neovascularisation;
GA: geographic atrophy;
L = London;
NMESES = North Midlands, East, Southeast and South of England;
NW = Northwest of England;
S = Scotland.
^{a}Controls were selected from the British 1958 Birth Cohort {Barrett, 2009 16; Power, 2006 17}.

TABLE 2
Association results for previously reported definite or probable AMD susceptibility loci in the discovery samples.

| | | | | | Effect | | | | | |
|---|---|---|---|---|---|---|---|---|---|---|
| | | | | | allele | | | | | |
| | | | | | (EA)/ | | | | | |
| | Reported SNP/proxy SNP | | | | Other | EA frequency | | | | |
| Locus | (R²) | Reference | Chr | Position | allele | Cases | Controls | P | OR | 95% CI |
| ARMS2- | rs10490924 | {Jakobsdottir, 2005 | 10 | 124204440 | T/G | 0.46 | 0.21 | 2.7 × 10⁻⁷² | 3.00 | 2.64-3.40 |
| HTRA1 | | 109; Rivera, 2005 | | | | | | | | |
| | | 106} | | | | | | | | |
| CFH | rs1061170/rs10801555 | {Klein, 2005 107} | 1 | 194926880 | G/A | 0.40 | 0.60 | 2.3 × 10⁻⁴⁷ | 0.43 | 0.38-0.48 |
| | (0.97) | | | | | | | | | |
| | rs1410996/rs1329428 (1.0) | {Maller, 2006 31} | 1 | 194969430 | T/C | 0.21 | 0.39 | 3.1 × 10⁻⁴³ | 0.40 | 0.35-0.45 |
| C2-CFB | rs641153/rs541862 (1.0) | {Gold, 2006 105} | 6 | 32024930 | T/C | 0.95 | 0.90 | 5.2 × 10⁻⁹ | 1.97 | 1.56-2.49 |
| | rs9332739 | {Maller, 2006 31} | 6 | 32011783 | G/C | 0.97 | 0.96 | 0.02 | 1.47 | 1.06-2.04 |
| VEGFA | rs4711751/rs943080 (1.0) | {Yu, 2011 35} | 6 | 43934605 | T/C | 0.54 | 0.49 | 1.6 × 10⁻³ | 1.20 | 1.07-1.35 |
| | rs833069 | {Galan, 2010 10} | 6 | 43850557 | T/C | 0.66 | 0.68 | 0.18 | 0.92 | 0.82-1.04 |
| C3 | rs2230199 | {Yates, 2007 1} | 19 | 6669387 | G/C | 0.76 | 0.79 | 2.2 × 10⁻³ | 0.80 | 0.69-0.92 |
| CCDC109B- | rs7690921 | {Neale, 2010 33} | 4 | 110787610 | T/C | 0.36 | 0.32 | 3.6 × 10⁻³ | 1.19 | 1.06-1.34 |
| PLA2G12A- | rs10033900 | {Fagerness, | 4 | 1108785200 | T/C | 0.51 | 0.49 | 0.22 | 1.07 | 0.96-1.20 |
| CFI | | 2009 38} | | | | | | | | |
| | rs2285714 | {Chen, 2010 34} | 4 | 110858259 | T/C | 0.45 | 0.46 | 0.92 | 0.99 | 0.89-1.11 |
| LIPC | rs493258 | {Chen, 2010 | 15 | 56475172 | T/C | 0.46 | 0.48 | 0.04 | 0.89 | 0.79-0.99 |
| | | 34; Neale, 2010 33} | | | | | | | | |
| | rs10468017 | {Yu, 2011 35} | 15 | 56465804 | T/C | 0.26 | 0.27 | 0.11 | 0.91 | 0.80-1.03 |
| CETP | rs3764261 | {Yu, 2011 35} | 16 | 55550825 | C/A | 0.66 | 0.67 | 0.26 | 0.93 | 0.83-1.05 |
| SYN3-TIMP3 | rs9621532 | {Yu, 2011 35} | 22 | 31414511 | C/A | 0.05 | 0.05 | 0.48 | 0.91 | 0.69-1.20 |

Chr: Chromosome;
Effect allele: allele for which the ORs and 95% CIs were calculated;
P: P-value from the stratified analysis using the Mantel's extension of the 1-df Cochrane-Armitage trend test;
Heterogeneity P-value for SNP rs10468017 = 0.03.

TABLE 3
GWAS and replication results for newly identified SNPs in TNXB, NOTCH4 and
other SNPs followed up in the replication samples.

| | | | | | | | English replication | |
|---|---|---|---|---|---|---|---|---|
| | | | | | Discovery samples | | samples | |
| | | | | | (893 cases, 2199 | | (1267 cases, 1102 | |
| | | | | Effect | controls) | | controls) | |
| | | | | allele | EA | | EA | |
| | | | | (EA)/ | frequency | | frequency | |
| SNP | Locus | Chr | Position | Other allele | (cases/controls) | P | (cases/controls) | P |

Newly identified SNPs

| | | | | | | | | |
|---|---|---|---|---|---|---|---|---|
| rs12153855 | TNXB | 6 | 32182782 | C/T | 0.023/0.011 | 4.3 × 10⁻⁷ | 0.016/0.008 | 6.8 × 10⁻⁵ |
| rs2071277 | NOTCH4 | 6 | 32279661 | G/A | 0.277/0.219 | 3.2 × 10⁻⁸ | 0.271/0.222 | 2.6 × 10⁻⁵ |

Other SNPs followed up for replication

| | | | | | | | | |
|---|---|---|---|---|---|---|---|---|
| rs541862 | CFB | 6 | 32024930 | G/A | 0.003/0.010 | 5.2 × 10⁻⁹ | 0.005/0.012 | 9.3 × 10⁻⁹ |
| rs3132946 | NOTCH4 | 6 | 32298006 | A/G | 0.009/0.019 | 2.7 × 10⁻³ | 0.024/0.019 | 0.7380 |
| rs13095226 | COL8A1 | 3 | 100878962 | C/T | 0.124/0.100 | 4.0 × 10⁻³ | 0.105/0.099 | 0.4807 |
| rs17778253 | COL8A1 | 3 | 100917792 | C/T | 0.113/0.084 | 4.3 × 10⁻⁴ | 0.095/0.087 | 0.3432 |
| rs12231166 | GOLGA2P5- | 12 | 20332348 | C/A | 0.164/0.116 | 6.2 × 10⁻⁷ | 0.134/0.135 | 0.9312 |
| | ACTR6 | | | | | | | |
| rs2075650 | TOMM40 | 19 | 50087459 | G/A | 0.105/0.149 | 3.1 × 10⁻⁶ | 0.007/0.022 | 5.6 × 10⁻⁴ |
| rs429358 | APOE | 19 | 50103781 | C/T | n/a | n/a | 0.006/0.024 | 3.4 × 10⁻⁷ |

| | Scottish replication | | | | |
|---|---|---|---|---|---|
| | samples | | | | |
| | (144 cases, 329 | | All samples combined | | |
| | controls) | | (2304 cases, 3630 controls) | | |
| | EA frequency | | | OR | |
| SNP | (cases/controls) | P | P | (95% CI) | Het P |
| Newly identified SNPs | | | | | |
| rs12153855 | 0.022/0.019 | 0.9266 | 1.3 × 10⁻⁹ | 1.44 | 0.3892 |
| | | | | (1.28-1.63) | |
| rs2071277 | 0.222/0.212 | 0.5481 | 2.0 × 10⁻¹¹ | 1.30 | 0.1869 |
| | | | | (1.12-1.41) | |
| Other SNPs followed up for replication | | | | | |
| rs541862 | 0.003/0 | 0.0777 | 8.8 × 10⁻¹⁷ | 0.53 | 0.9112 |
| | | | | (0.46-0.62) | |
| rs3132946 | 0.028/0.022 | 0.9694 | 2.9 × 10⁻² | 0.88 | 0.4089 |
| | | | | (0.79-0.99) | |
| rs13095226 | 0.128/0.113 | 0.5205 | . . . | . . . | . . . |
| rs17778253 | 0.112/0.098 | 0.5501 | . . . | . . . | . . . |
| rs12231166 | 0.135/0.127 | 0.7379 | . . . | . . . | . . . |
| rs2075650 | 0.016/0.006 | 0.2288 | . . . | . . . | . . . |
| rs429358 | 0.008/0.003 | 0.7469 | 2.8 × 10⁻⁶ | 0.68 | 0.0414 |
| | | | | (0.58-0.80) | |

Chr: Chromosome;
P: P-value from the stratified analysis using the Mantel's extension of the 1-df Cochrane-Armitage trend test;
Het P: Heterogeneity P-value.
For the GOLGA2P5-ACTR6 locus, proxy SNP rs476497 (R² = 0.92) was followed up in the replication samples. Het P for SNP rs2075650 = 0.02 in the replication samples combined.

TABLE 4
Conditional association analysis in the extended C2/CFB region.

| | | | | | Discovery samples | | Replication samples | | All samples combined | |
|---|---|---|---|---|---|---|---|---|---|---|
| | | | | | (893 cases, 2199 controls) | | (1411 cases, 1431 controls) | | (2304 cases, 3630 controls) | |
| | | | | Conditioning | Unconditioned | Conditioned | Unconditioned | Conditioned | Unconditioned | Condi- |
| Test SNP | Gene | Chr | Position | SNP(s) | P | P | P | P | P | tioned P |
| rs541862 | CFB | 6 | 32024930 | rs541862 | 4.9 × 10⁻⁹ | n/a | 2.2 × 10⁻⁹ | n/a | 8.8 × 10⁻¹⁷ | n/a |
| rs12153855 | TNXB | 6 | 32182782 | | 4.2 × 10⁻⁷ | 9.0 × 10⁻⁶ | 2.9 × 10⁻⁴ | 0.0024 | 1.3 × 10⁻⁹ | 1.6 × 10⁻⁷ |
| rs2071277 | NOTCH4 | 6 | 32279661 | | 3.1 × 10⁻⁸ | 2.3 × 10⁻⁵ | 3.8 × 10⁻⁵ | 0.0048 | 2.0 × 10⁻¹¹ | 1.1 × 10⁻⁶ |
| rs3132946 | NOTCH4 | 6 | 32298006 | | 2.7 × 10⁻³ | 4.9 × 10⁻⁴ | 0.77 | 0.32 | 0.03 | 2.5 × 10⁻³ |
| rs541862 | CFB | 6 | 32024930 | rs541862, | 4.9 × 10⁻⁹ | n/a | 2.2 × 10⁻⁹ | n/a | 8.8 × 10⁻¹⁷ | n/a |
| rs12153855 | TNXB | 6 | 32182782 | rs12153855 | 4.2 × 10⁻⁷ | n/a | 2.9 × 10⁻⁴ | n/a | 1.3 × 10⁻⁹ | n/a |
| rs2071277 | NOTCH4 | 6 | 32279661 | | 3.1 × 10⁻⁸ | 3.8 × 10⁻³ | 3.8 × 10⁻⁵ | 0.05 | 2.0 × 10⁻¹¹ | 9.1 × 10⁻⁴ |
| rs3132946 | NOTCH4 | 6 | 32298006 | | 2.7 × 10⁻³ | 3.5 × 10⁻³ | 0.77 | 0.58 | 0.03 | 0.02 |
| rs541862 | CFB | 6 | 32024930 | rs541862, | 4.9 × 10⁻⁹ | n/a | 2.2 × 10⁻⁹ | n/a | 8.8 × 10⁻¹⁷ | n/a |
| rs12153855 | TNXB | 6 | 32182782 | rs2071277 | 4.2 × 10⁻⁷ | 2.0 × 10⁻³ | 2.9 × 10⁻⁴ | 0.04 | 1.3 × 10⁻⁹ | 2.5 × 10⁻⁴ |
| rs2071277 | NOTCH4 | 6 | 32279661 | | 3.1 × 10⁻⁸ | n/a | 3.8 × 10⁻⁵ | n/a | 2.0 × 10⁻¹¹ | n/a |
| rs3132946 | NOTCH4 | 6 | 32298006 | | 2.7 × 10⁻³ | 0.04 | 0.77 | 0.95 | 0.03 | 0.21 |
| rs541862 | CFB | 6 | 32024930 | rs541862, | 4.9 × 10⁻⁹ | n/a | 2.2 × 10⁻⁹ | n/a | 8.8 × 10⁻¹⁷ | n/a |
| rs12153855 | TNXB | 6 | 32182782 | rs3132946 | 4.2 × 10⁻⁷ | 7.7 × 10⁻⁵ | 2.9 × 10⁻⁴ | 0.003 | 1.3 × 10⁻⁹ | 1.2 × 10⁻⁶ |
| rs2071277 | NOTCH4 | 6 | 32279661 | | 3.1 × 10⁻⁸ | 2.2 × 10⁻³ | 3.8 × 10⁻⁵ | 0.01 | 2.0 × 10⁻¹¹ | 1.4 × 10⁻⁴ |
| rs3132946 | NOTCH4 | 6 | 32298006 | | 2.7 × 10⁻³ | n/a | 0.77 | n/a | 0.03 | n/a |
| rs541862 | CFB | 6 | 32024930 | rs541862, | 4.9 × 10⁻⁹ | n/a | 2.2 × 10⁻⁹ | n/a | 8.8 × 10⁻¹⁷ | n/a |
| rs12153855 | TNXB | 6 | 32182782 | rs12153855, | 4.2 × 10⁻⁷ | n/a | 2.9 × 10⁻⁴ | n/a | 1.3 × 10⁻⁹ | n/a |
| rs2071277 | NOTCH4 | 6 | 32279661 | rs3132946 | 3.1 × 10⁻⁸ | 0.06 | 3.8 × 10⁻⁵ | 0.09 | 2.0 × 10⁻¹¹ | 0.01 |
| rs3132946 | NOTCH4 | 6 | 32298006 | | 2.7 × 10⁻³ | n/a | 0.77 | n/a | 0.03 | n/a |
| rs541862 | CFB | 6 | 32024930 | rs541862, | 4.9 × 10⁻⁹ | n/a | 2.2 × 10⁻⁹ | n/a | 8.8 × 10⁻¹⁷ | n/a |
| rs12153855 | TNXB | 6 | 32182782 | rs12153855, | 4.2 × 10⁻⁷ | n/a | 2.9 × 10⁻⁴ | n/a | 1.3 × 10⁻⁹ | n/a |
| rs2071277 | NOTCH4 | 6 | 32279661 | rs2071277 | 3.1 × 10⁻⁸ | n/a | 3.8 × 10⁻⁵ | n/a | 2.0 × 10⁻¹¹ | n/a |
| rs3132946 | NOTCH4 | 6 | 32298006 | | 2.7 × 10⁻³ | 0.04 | 0.77 | 0.92 | 0.03 | 0.22 |

Chr: Chromosome;
P: P-value from the stepwise logistic regression using the Wald test on the beta coefficients;
Linkage Disequilibrium based on HapMap II: R²(rs541862, rs12153855) = 0.01, R²(rs541862, rs2071277) = 0.02, R²(rs541862, rs3132946) = 0.02, R²(rs12153855, rs2071277) = 0.14, R²(rs12153855, rs3132946) = 0.02, R²(rs2071277, rs3132946) = 0.17.

TABLE 5
Association analysis for the common haplotypes spanning CFB-TNXB-NOTCH4.

| | | | Discovery | | Replication | | All samples combined | | | |
|---|---|---|---|---|---|---|---|---|---|---|
| | | | samples | | samples | | (2304 cases, 3630 controls) | | | |
| Haplotype | | | (893 cases, | | (1411 cases, | | Haplotype | | | |
| rs541862 | rs12153855 | rs2071277 | 2199 controls) | | 1431 controls) | | frequency | | | |
| (CFB) | (TNXB) | (NOTCH4) | OR | 95% CI | OR | 95% CI | Cases | Controls | OR | 95% CI |
| T | T | T | 1 | | 1 | | 0.423 | 0.449 | 1 | |
| T | T | C | 1.18 | 1.04-1.34 | 1.13 | 1.00-1.27 | 0.394 | 0.359 | 1.14 | 1.05-1.25 |
| T | C | C | 1.62 | 1.35-1.94 | 1.35 | 1.12-1.62 | 0.132 | 0.098 | 1.47 | 1.29-1.67 |
| C | T | T | 0.54 | 0.42-0.71 | 0.58 | 0.46-0.73 | 0.051 | 0.094 | 0.56 | 0.48-0.67 |

ATA, ATG, ACG and GTA account for 98.9% of the total haplotypes observed.

### ABBREVIATIONS

AGER: Advanced glycosylation end product-specific receptor
AMD: Age-related macular degeneration
APOE: Apolipoprotein E
ARMS2: Age-related maculopathy susceptibility 2
C2: Complement component 2
C3: Complement component 3
CCDC109B: Coiled-coil domain containing 109B
CETP: Cholesteryl ester transfer protein
CFB: Complement factor B
CFH: Complement factor H
CFI: Complement factor I
CI: Confidence interval
CNV: Choroidal neovascularisation
FKBPL: FK506 binding protein like
GA: Geographic atrophy
GWAS: Genome wide association study
HTRA1: HtrA serine peptidase 1
LD: Linkage disequilibrium
LIPC: Hepatic lipase
NOTCH4: Notch 4
OR: Odds ratio
PLA2G12A: Phospholipase A2, group XIIA
RDBP: RD RNA binding protein
RPE: Retinal pigment epithelium
SKIV2L: Superkiller viralicidic activity 2-like
SNP: Single nucleotide polymorphism
SYN3: Synapsin III
TIMP3: TIMP metallopeptidase inhibitor 3
TNXB: Tenascin XB
TOMM40: Translocase of outer mitochondrial membrane 40 homolog
VEGFA: Vascular endothelial growth factor A
VEGFB: Vascular endothelial growth factor B

1. A method for identifying a risk profile for AMD or CNV in a subject, comprising identifying the nucleotide at one or more of the following positions: rs2071277, rs12153855 and rs9391734, or a proxy of any of those sites, in a sample obtained from the subject.

2. The method according to claim 1, wherein the method comprises the step of identifying the nucleotide at position rs2071277 and/or one of rs12153855 and rs9391734.

3. The method according to claim 1, wherein the method also comprises the step of identifying the nucleotide at position rs541862.

4. The method according to claim 1, further comprising identifying a nucleotide at any other sites known to be associated with AMD or CNV.

5. An isolated nucleic acid consisting of between about 15 and 2000 contiguous nucleotides from the sequence shown in any one of:
FIG. 2 (SEQ ID NO:1) and which includes position rs12153855;
FIG. 3 (SEQ ID NO:2) and which includes position rs9391734; and
FIG. 4 (SEQ ID NO:3) and which includes position rs2071277; or
an isolated nucleic acid which hybridises to part or all of a polynucleotide consisting of between about 15 and 2000 contiguous nucleotides from the sequence shown in any one of:
FIG. 2 (SEQ ID NO:1) and which includes position rs12153855;
FIG. 3 (SEQ ID NO:2) and which includes position rs9391734; and
FIG. 4 (SEQ ID NO:3) and which includes position rs2071277.

6-10. (canceled)

11. The isolated nucleic acid according to claim 5, wherein the isolated nucleic acid is between 8 and 100 bases in length.

12. A kit for identifying a risk profile of AMD or CNV in a subject, the kit comprising one or more isolated nucleic acids according to claim 5.

13. A kit for identifying a risk profile of AMD or CNV in a subject, the kit comprising
an isolated nucleic acid selected from
an isolated nucleic acid consisting of between about 15 and 2000 contiguous nucleotides from the sequence shown in FIG. 4 (SEQ ID NO:3) and which includes position rs2071277, and
an isolated nucleic acid which hybridises to part or all of a polynucleotide consisting of between about 15 and 2000 contiguous nucleotides from the sequence shown in FIG. 4 (SEQ ID NO:3) and which includes position rs2071277; and
an isolated nucleic acid selected from
an isolated nucleic acid consisting of between about 15 and 2000 contiguous nucleotides from the sequence shown in FIG. 2 (SEQ ID NO:1) and which includes position rs12153855 or FIG. 3 (SEQ ID NO:2) and which includes position rs9391734, and
an isolated nucleic acid which hybridises to part or all of a polynucleotide consisting of between about 15 and 2000 contiguous nucleotides from the sequence shown in FIG. 2 (SEQ ID NO:1) and which includes position rs12153855 or FIG. 3 (SEQ ID NO:2) and which includes position rs9391734.

14. (canceled)

15. The method according to claim 2, wherein the method also comprises the step of identifying the nucleotide at position rs541862.

16. The method according to claim 2, further comprising identifying a nucleotide at any other sites known to be associated with AMD or CNV.

17. The method according to claim 3, further comprising identifying a nucleotide at any other sites known to be associated with AMD or CNV.


SEQUENCE LISTING (3 sequences)

SEQ ID NO: 1 - DNA, 2001 bp, Homo sapiens
tgcacagttctatttatgtgaagtttgaggagaggcaacattaatctgtagtgctccaagtcagaagtgtgatactcttgagggggtagtgactagtaggggcatgggggggggggcttctggggtgctaaaggtgtcttgtttcttgatctggatgacggttacccagttgtatgtgtttgtggagtttactgaggcagggattggggagcacacagagacaagaggtgggaggaaaggtggtaggggggtcccaggataagacaggagacagggctatggcaaacctgagtgtgagggggccagtcaggattcagctagggtgaagagttgaggagagatgttgaaaggtgcccagctcttccagaccagaggctggggaaaggaggagggcagggtgggtgtggaaatgctgaccggtgaggagtgggggatgtgtcactggtaggcggaggtgactgtgacacacacacacctccacccaacacgcacacatcttctggctttctactcttaaacttttctcctggatcagagaaaaaggagtaattggggctaccagccatcacattctgctaccagccagcaagagacggaaagggagctgaacagggacagagtgtttccacaccaagaggctccccacccaagaaagcccaggccgagaggcctggcatacagagagctgccctttcctgtctccccagcccttctctcagcctggagtgtagccttgggtaagagatgggctctgaccgaccctcaaatcctgtcactttgtgtctaaggccatgctaatcactcccacaccctgggaatgcttgcacaaagatgtgtacacatgtacacttacaaatatattaatgtgtgctcatccacaagtacaaacatacctgcacagacacacaaatccacccccaaaacctgcatccttgtaggtacacatatgggtgcccatgcacacacgtgcacacacacccaatctccccttcaggtttccyccacacctagtcattgggtgatgcctaagaaggctgatttcttgttggctccgagggcagctctgtgagtgaaagaacctggatggtgtgagaggaacaaagcaggaggcccttcccagtgggaagcagcggacacatggcctccaccatacttccccgtacccctggaggcctctgtgagttgtccagtgcccctccccaaataagtggattcctcatctttccctgcacagtgggggaaagcagagagggtctttgcaccaggaaggagtgaagagagctcaccagcttttctggaaagctggtgagattggtctagaagagcagagcagcctccattgaccacacacccagaggctccttcccaatctccctccctgccctgttgccaccacccagccctccagctccccattccagctcctgggataattctgctcttctctcggtactgggcaagcaaagaattgggtgtctcttgtgtctccccttcaccattccccctgccactatgtctggggtggttctctgtagtgtgtgtttggagagagatatggcctcaccctcttggcaccctccccacagttggagggctggcagggatgggggcatcttcccaaactgtgactcagcttcctgggtattttgtgttgtagccaactttgacactttgctcattagggactgtataggtcccaaggggtggggagaggggctggcttccataaaagatgttccacagatgggggttctggtcctgcccccatgcctccctctgaccctcctgctacaccctcagttgaatctggcaatgagaggtgaaggtagaaggattctaggagtgagactaaaaagggaatgtgggggtctcagatattgggctggaaccaggtaggaaggaggtgtcagggaggggtaggttggggaatccagataccccttattctggggagctaatcggcctgggaatggaaaaattaagagaaaggatttccaggcc

SEQ ID NO: 2 - DNA, 2001 bp, Homo sapiens
aaggatgccagtgtgagcctgacaggaggtccagagtgcccaggcagctgaagctctgcttcctcaccttgacacatggactccaagcatttctctatgagctccccccaagtttcctccctccatggccctacgcccatagttagctctgtccagccctctggcggccctgatccgaaaggaaaccccaaagccagtacccggcagcaggagcctagtttgggtttgtccaagccatggccacggattacgatcttcttgacaaagctcccatcggggcagtaccagagatcagaagcttgaagggcctctggcatctgactggttgatccatgagacttatgagagtctccttcaagttcagcaaccagtttttcagctccttgagtatgctctagaatttggctggctggatctgggcttacttccagctcaagcgtttcggtaggagggtctcggggctgctgcctaatctgaataactgaatcaaggttctcccgaaggttcttttcccactcttgttgcggctgagaggtgtccttttctccaattgtattgactggtggcgtctccatatggatgcttagtcccttccacggtgagtgaacagttttggtcagaaagggatgaaccaggttcaggtcagcacctgaaaagaaaaccaaacaatgctaatagcagggttcttatttagactcctttctcgtcctttcccattcttctgagacccaggcccctagtcctgaaagtccccagttttgctttcctccaaaaatctgctccagcttccccgttccacccacatataatttagaactataaattccacaattccctgcggttaaggtagccgcgccaactacggacacccggtcgggtcaataagtacctgcgcggccaaagtgcctagcatggtgacaggaggagccgggccattcgaatcacctctccttccaaagctaaatggctactgaatgctgccctcggagccttgccccacrcggagagggcagccggagaggggcgcggtgcgggaggcgggggtagggggcggaacaactgggaaagatactgacaactaaccctggagcccgcgagactccgaatctagtcaaattcctggcagccaatcgggagaagggaggaatctggttagcccgcctattgaacgtgacatcatttcctccgcaaccatgaagctccaggccttagcaactgaactaggccagagcaaccgaactaggcagaatcgaacagaatttggcgcggtcgggctggccaggctgctcaagagtcaaagtgggccaacatggtgagaccctgtctctactaaaaatacaaaaattagtaggtcatggtggcgcgcgcctgtaatcccaactactcggaaggctgaaacaggagaatcgcttgaacccgggagacagacgttgcagtgagccgagatcacaccattgcactccagcctgggcgacagagcgagactagagactccgtctcaaacaaaaaaaaaaaaaaaaaaaaaaaaaaaaggtgggccgggcgcggtggctcacgcctgtaatcccagcactttgggaggttgaggtcaggagttcaagaccagcctggccaacatggtgaaaccccgtctctactaaaaatgcaaaaatagctcggcgtgatggcgggcgcctatatcccagctacccaggaggctgaggcaggagaatggcttacctgggaagcggaggttgcagtgagccgagatcgcgccattgcactccagctttggcaactgagactctgggaggctgaggtgggcggatcacgaggtcaggtgatcgagaccatcctggcaacatggtgaaatctcgtctctactaaaaatacaaaaagttagctgggcatggtggcgtgtgcctgtaatcccagctacttgggaggctgaggtgggagaatcgcttgaaccagggagtcggaggttgcagtgagccgagatggtgccactgcaatccagcc

SEQ ID NO: 3 - DNA, 2001 bp, Homo sapiens
ttattattattatttttgagacagagtttcgctcttgttgtccaggctggagtgcaatggtgcaatctcagctcattgcaacctctgcctcccgggttcaagcaattctcctgcctcagccttcccaatagctgggattacagccatgcaccaccacgcccagctaatttgtatttttagtagagacagggtttctccatgtttgtcaggctggtctcgaatgcccgacctcaggtgatccgcctgcctcggcctcccaaagtgctgggattacaggcgtgagccactgtgcccggccggctgttatattattatcttactccttagaaataggatcatatgtcttcctcttcctctggagagggaacaggatacaggaggaggacttaagtagatgtaagttattattaatattgaaattcttgggttaggttcatgggtgttacattgttagaataataaaataaaagaagaccaggcataaaccaatgtcagtgtatcaggaaccaaagcttaagattagtctaattccatgcatctgaggtccataaatacatatacaaacacacacagagttaaaataacctatctgaggccacccaccacgcagcttgagcttggggagctcctgaccttcccttaggcaacgcctgtgatttttgaaaattccattcatgctatcaactgatcctgccttgcctttgactgcttctgagagacacttcccactgtgagcttggcatggctttttccaataatttccacatcagtgctcacccacagtcccttctgggattccaactgaggtattcttgccttgtcagcatagggggcaacagagaaggcagatttgtggtcacttgccttggggcagtggcagatgaaacccaggggtgatcctgctgtggcctcacaggtccctccatgaaagcagggttggctgtggcaggggtctatctccacctcacaccactggcctgtaattatgggggagattagaygtcacacactgcatcagtcactgcctccatcctagctcattcctggatgttggcccagtgctagatgtgcaggtgaagggatcctggggcatcttttctgggcgggggtgggcgtggaggcaggggatggaccaggtgacggctgccgcatgggtggagactatctggctctccatggtctgcttggctgtgctccagacacacttgtgccccttgtcttggggcctcacctgtgtgtccaggcagacactggcagtagaaggcattggccagagagtggcaggctgcagtgcctgtggggtggcagggctggtccagacactcgtccacgtctccctcacagcgtagccccacaaagcctggagggcaggcacagtggaatcctccaggtttgggagtacaggttccatggttgtgacagggttgggactgacaagcatcgagttcctttgagcagttctgtccatcgtagcctggggcacactgcagacaaagaggattagacagggaaccagtggatgagcccaacccagcactacaagggacccagctcaagatagtctgtccagtcccccaccttccagctcaacagcatcactcaactcaccatccatcatggccatgtgtcacaatccttctatctcaactccccatgagacacaattgttggcgacacacaactcaaacttccccagtcccaaacaatctctatgacacactgccaccaaacacagcaccatttttggtaaaaccttcctcccctgctaaatacctaccaggctctctcatactttattaattcataagcatctattgagtgcctactttgtgtcaggcaccgttttaggcactaggaatacaaagaaagttagaacccattcctatttcctggaagctctcagtcaaccagaggaaagaaatgactagcatttattgcatgatttatatacataacctaaaaatccccctaatgacattttatttgggttatctcattcga